(19) United States
(12) Patent Application Publication
(10) Pub. No.: US 2012/0189578 A1
(43) Pub. Date: Jul. 26, 2012

(54) COMPOSITIONS AND METHODS FOR POTENTIATING INTERLEUKIN-35

(75) Inventors: Lauren W. Collison, Memphis, TN (US); Dario A.A. Vignali, Germantown, TN (US)

(73) Assignee: St. Jude Children's Research Hospital, Memphis, TN (US)

(21) Appl. No.: 13/389,106

(22) PCT Filed: Aug. 13, 2010
(86) PCT No.: PCT/US2010/045420

§ 371 Date: Apr. 11, 2012

Related U.S. Application Data
(60) Provisional application No. 61/236,378, filed on Aug. 24, 2009.

(51) Int. Cl. A61K 38/20; G01N 33/566; C12N 5/0783

(52) U.S. Cl. 424/85.2; 435/375; 436/501

(57) ABSTRACT

Compositions and methods are provided for potentiating activity of interleukin-35 (IL-35). Such compositions and methods include administering therapeutically effective amounts of non-blocking IL-35 binding agents. The non-blocking IL-35 binding agents do not block the binding of IL-35 to its target(s). Also provided are methods to identify non-blocking IL-35 binding agents that enhance IL-35 activity.

This invention was made with United States Government support under NIH R01 Grant A139480 awarded by the National Institute of Health. The United States Government has certain rights in the invention.

### FIELD OF THE INVENTION

The invention relates generally to compositions and methods for potentiating interleukin-35 (IL-35) activity, and more particularly to compositions and methods for potentiating IL-35 activity by use of a non-blocking IL-35 binding agent.

### BACKGROUND OF THE INVENTION

Regulatory T (T_{reg}) cells are a sub-population of CD4⁺ T cells that maintain self tolerance and prevent autoimmunity, that limit chronic inflammatory diseases such as asthma and inflammatory bowel disease, and that regulate homeostatic lymphocyte expansion. They also, however, can suppress natural immune responses to parasites and viruses and can suppress anti-tumor immunity induced by therapeutic vaccines. Collison et al. (2007) Nature 450:566-569. Molecules that mediate T_{reg} cells' suppressive activity are largely unknown.

Collison et al. recently demonstrated that T_{reg} cells, but not resting or activated CD4⁺ effector T (T_{eff}) cells, express and secrete IL-35. Collison et al., supra. IL-35 is a member of the interleukin-12 (IL-12) cytokine family and is an inhibitory, heterodimeric cytokine having an α chain (a p35 subunit of IL-12a) and a β chain (an Epstein Barr virus induced gene 3 (EBI3; IL27b) subunit). Devergne et al. (1997) Proc. Natl. Acad. Sci. USA 94:12041-12046. Collison et al. also demonstrated that ectopic expression of IL-35 conferred regulatory activity on naïve T cells and that recombinant IL-35 suppressed T cell proliferation. Collison et al., supra.

To produce its suppressive effects, IL-35 selectively acts on different T cell subset populations. For example, IL-35 expands T_{reg} cells, but suppresses proliferation of T_{eff} cells (e.g., Tₕ17 cells). Niedbala et al. (2007) Eur. J. Immunol. 37:3021-3029. IL-35 also suppresses inflammatory responses of other immune cells (e.g., dendritic cells, macrophages, natural killer cells, etc.). As such, IL-35 is one molecule believed to mediate T_{reg} cells' suppressive activity and to assist T_{reg} cells in immune suppression, immune system homeostasis and tolerance to self-antigens.

Given the important role of IL-35 in immune suppression, immune system homeostasis and tolerance to self-antigens, a need exists for agents that potentiate IL-35's activity.

### BRIEF SUMMARY OF THE INVENTION

The present invention broadly relates to compositions and methods for potentiating IL-35's activity by use of a non-blocking IL-35 binding agent. The non-blocking IL-35 binding agents do not block the binding of IL-35 to its target(s). The compositions include non-blocking IL-35 binding agents alone or in combination with exogenous IL-35. The methods include contacting an effective amount of non-blocking IL-35 binding agent with IL-35. Optionally, the methods include providing exogenous IL-35. Also included are methods to identify non-blocking IL-35 binding agents that enhance IL-35 activity.

These and other features, objects and advantages of the present invention will become better understood from the description that follows. In the description, reference is made to the accompanying drawings, which form a part hereof and in which there is shown by way of illustration, not limitation, embodiments of the invention. The following description is not intended to limit the invention to cover all modifications, equivalents and alternatives. Reference should therefore be made to the claims recited herein for interpreting the scope of the invention.

### BRIEF DESCRIPTION OF THE DRAWINGS

The present invention will be better understood and features, aspects and advantages other than those set forth above will become apparent when consideration is given to the following detailed description thereof. Such detailed description makes reference to the following drawings, wherein:

FIGS. 1A-B show that EBI3 and p35 antibodies enhance IL-35-mediated suppression of T_{eff} proliferation. FIG. 1A shows suppression of T_{eff} cells by anti-EBI3 antibodies. In FIG. 1A, FACS-purified T_{eff} cells (2.5×10⁴/well) were pre-incubated with indicated antibodies at 10 μg/ml for 10 minutes at 37° C. Following antibody treatment, T_{eff} cells were activated with anti-CD3- and anti-CD28-coated sulfate latex beads at 5×10³/well in the presence of dialyzed, filtered HEK293T supernatant containing rIL-35. Proliferation was determined by [³H]-thymidine incorporation. FIG. 1B shows suppression of T_{eff} by anti-EBI3 and anti-p35 antibodies. In FIG. 1B, T_{eff} cells (2.5×10⁴/well) were activated as described in FIG. A in the presence of rIL35 containing supernatant and antibodies at indicated concentrations. IgG1 and IgG2 isotype controls were used to determine specificity. Proliferation was determined by [³H]-thymidine incorporation.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

The present invention relates to an identification of non-blocking IL-35 binding agents, such as anti-IL-35 antibodies, which increase activity of IL-35. The agents can be provided in vivo or in vitro to potentiate IL-35's activity, thereby affecting T_{reg} and T_{eff} cell function. When provided in vivo, the agents potentiate IL-35's ability to suppress the immune system and to attenuate an autoimmune or inflammatory condition. While not intending to be bound to any particular theory, the non-blocking IL-35 binding agents may potentiate IL-35's signal, increase its half-life (t½) or both. As such, compositions and methods for potentiating IL-35 activity are described. The compositions comprise non-blocking IL-35 binding agents that act as agonists and that are specific for IL-35 or its subunits.

As used herein, “interleukin-35” or “IL-35” means any intramolecular complex or single molecule comprising at least one EBI3 polypeptide component and at least one p35 polypeptide component. See, e.g., Int'l Patent Application Publication No. WO 2008/036973; incorporated herein by reference as if set forth in its entirety. IL-35 also encompasses naturally occurring variants (e.g., splice variants, allelic variants and other known isoforms), as well as fragments or variants of IL-35 that are active and bind its target(s).

EBI3 and p35 are known in the art. Nucleic and amino acid sequences for EBI3 are known. See, e.g., GenBank Accession Nos. BC046112 (human EBI3) and NM_{—}015766 (mouse EBI3). Likewise, nucleic and amino acid sequences for p35 are also known. See, e.g., GenBank Accession Nos. NM_{—}000882 (human p35) and M86672 (mouse p35); see also, Int'l Patent Application Publication No. WO 97/13859; incorporated herein by reference as if set forth in its entirety. In vivo, EBI3 and p35 typically associate via a non-covalent association.

The compositions and methods described herein are useful in a variety of applications. For example, the compositions and methods can be used to treat a subject having or susceptible to having an autoimmune condition. That is, a subject having type 1 diabetes can be administered an IL-35 binding agent or pre-formed IL-35/IL-35 binding agent complex to suppress autoimmune destruction of insulin-producing beta cells of the islets of Langerhans in the pancreas. Alternatively, the compositions and methods can be used to treat a subject having or suspected of having an inflammatory condition. That is, a subject having or susceptible to having asthma can be administered the non-blocking IL-35 binding agent or pre-formed IL-35/IL-35 binding agent complex to attenuate a mixed cellular infiltrate dominated by T_{eff} cells that are often responsible for epithelial damage and mucus hypersecretion. Moreover, the methods described herein can be used to discover additional non-blocking IL-35 binding agents.

Unless defined otherwise, all technical and scientific terms used herein have the same meaning as commonly understood by one of ordinary skill in the art to which the invention pertains. Many modifications and other embodiments of the inventions set forth herein will come to mind to one of ordinary skill in the art having the benefit of the teachings presented in the foregoing description and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Compositions

The present invention includes compositions having at least an effective amount of a non-blocking IL-35 binding agent. As used herein, a “non-blocking IL-35 binding agent” means an agent that binds substantially only to IL-35, but does not block IL-35's ability to bind to its target(s). As used herein, “binds substantially only to” means that the non-blocking IL-35 binding agent binds to a subunit (i.e., EBI3 or p35) of IL-35 or to IL-35 itself and/or potentiates activity of IL-35. Moreover, the non-blocking IL-35 binding agent that binds substantially only to a subunit of IL-35 or IL-35 itself should not complex with other cytokines or cytokine combinations, such as IL-12 or IL-27, as IL-35 shares subunits with IL-12 (p35) and IL-27 (EBI3).

In one embodiment, a composition for potentiating IL-35 is provided, which comprises an effective amount of a non-blocking IL-35 binding agent that enhances IL-35 activity. In other embodiments, a pharmaceutical composition for potentiating IL-35 is provided, which comprises a therapeutically effective amount of a non-blocking IL-35 binding agent and a pharmaceutically acceptable carrier. In either embodiment, the non-blocking IL-35 binding agent binds substantially only to IL-35, but does not block IL-35's ability to bind to its target(s).

In some embodiments, the non-blocking IL-35 binding agent can be an anti-IL-35 antibody, a protein designed to bind IL-35 or a small molecule that specifically binds to IL-35. When the non-blocking IL-35 binding agent is an antibody, it can be a monoclonal antibody and can bind a p35 or EBI3 subunit of IL-35 or IL-35 itself. When the non-blocking IL-35 binding agent is a small molecule, it can be a chemical compound and can bind a p35 or EBI3 subunit of IL-35 or IL-35. The non-blocking IL-35 binding agent also can be an IL-35/non-blocking IL-35 binding agent complex.

As used herein, “potentiating activity of IL-35,” “potentiate activity of IL-35,” “potentiating IL-35's activity” or “potentiate IL-35's activity” means any statistically significant increase in IL-35 activity. Such an increase in IL-35 activity can be measured by a variety of methods known in the art, such as measuring increased ability to expand T_{reg} cells, to reduce activity of T_{eff} cells, to suppress inflammatory responses of other immune cells, and/or to increase the half-life of IL-35. An ability of the non-blocking IL-35 binding agent to potentiate activity of IL-35 can be measured by any method known in the art for assaying IL-35 activity, such as the methods described in greater detail below. Alternatively, the ability of the non-blocking IL-35 binding agent to potentiate activity of IL-35 can be measured by any method known in the art for assaying T_{reg} and/or T_{eff} cell function.

Regardless of the exact nature of the non-blocking IL-35 binding agent, it enhances one or more of IL-35's activities. The activity increases by a statistically significant amount including, but not limited to, about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% of IL-35′s activity compared to an appropriate control. Conversely, the non-blocking IL-35 binding agent should not statistically decrease IL-35's activity.

As used herein, an “effective amount” or “therapeutically effective amount” (i.e., dosage) means an amount of the non-blocking IL-35 binding agent provided in vitro or in vivo, respectively, sufficient to contact and operably complex (either covalently or non-covalently) with IL-35 or one of its subunits, for which it has binding specificity, and to potentiate IL-35's activity. Moreover, the effective amount or therapeutically effective amount of the non-blocking IL-35 binding agent is the amount that is sufficient to achieve a desired effect, such as increasing IL-35 t½, enhancing immune suppression, promoting T_{reg} cell expansion or inhibiting/attenuating a T_{eff} cell function. For example, this can be the amount of the non-blocking IL-35 binding agent useful in preventing or overcoming various immune disorders such as arthritis, allergy or asthma. The therapeutically effective amount of the non-blocking IL-35 binding agent will depend on the subject being treated, the severity of the disorder and the manner of administration. Alternatively, this can be the amount that would saturate (e.g., bind substantially all available) any specific and available non-blocking binding sites of IL-35. Alternatively still, this can be the amount that would achieve a target tissue concentration similar to that which produces a desired effect in vitro.

The therapeutically effective amount of the non-binding IL-35-specific binding agent can be determined by in vitro or in vivo animal studies. The therapeutically effective amount (i.e., dosage) is administered to the subject to provide a target tissue concentration similar to that which has been shown to be effective in the animal assays. It is contemplated that genetically modified animals may be useful for exaggerating a potentiated IL-35 signal. Examples of genetically modified animals include, but are not limited to, p40^{-/-}, p35^{-/-}, EBI3^{-/-} animals and the like. See, e.g., Collison & Vignali (2008) Immunol. Rev. 226:248-262; incorporated herein by reference as if set forth in its entirety.

Generally, the therapeutically effective amount can be from about 0.0001 mg/kg to about 1000 mg/kg of body weight in the treatment of immune system disorders, alternatively, from about 0.001 mg/kg to about 900 mg/kg of body weight of the subject, from about 0.01 mg/kg to about 800 mg/kg, from about 0.1 mg/kg to about 700 mg/kg, from about 1.0 mg/kg to about 600 mg/kg, from about 10 mg/kg to about 500 mg/kg, from about 100 mg/kg to about 400 mg mg/kg or from about 200 mg/kg to about 300 mg/kg of body weight. Alternatively still, the therapeutically effective amount can be about 0.001 mg/kg to about 0.01 mg/kg, about 0.01 mg/kg to about 0.1 mg/kg, about 0.1 mg/kg to about 1 mg/kg, about 1 mg/kg to about 10 mg/kg, about 10 mg/kg to about 100 mg/kg, about 100 mg/kg to about 1000 mg/kg or more of body weight.

Stated differently, the therapeutically effective amount can be from about 0.01 mg to about 100 g per subject in the treatment of immune system disorders, alternatively, from about 0.1 g to about 90 g, from about 1 g to about 80 g, from about 10 g to about 70 g, from about 20 g to about 60 g or from about 30 g to about 50 g per subject. Alternatively still, the therapeutically effective amount can be from about 100 g, 95 g, 90 g, 85 g, 80 g, 75 g, 70 g, 65 g, 60 g, 55 g, 50 g, 45 g, 40 g, 35 g, 30 g, 25 g, 20 g, 15 g, 10 g, 5 g, 1 g, 0.1 g, 0.01 g, 0.001 g, 0.0001 g or 0.00001 g per subject. For example, type I diabetes mellitus can be effectively treated by the administration from about 0.01 mg to about 100 mg of the non-blocking IL-35 binding agent per kg of body weight, or alternatively from about 0.5 mg to about 10 g per subject.

As used herein, “about” means within a statistically meaningful range of a value such as a stated concentration range, time frame, molecular weight, temperature or pH. Such a range can be within an order of magnitude, typically within 20%, more typically still within 10%, and even more typically within 5% of a given value or range. The allowable variation encompassed by the term “about” will depend upon the particular system under study, and can be readily appreciated by one of ordinary skill in the art.

The methods can comprise administering therapeutically effective amounts of multiple doses of a non-blocking IL-35 binding agent. A therapeutically effective amount of the non-blocking IL-35 binding agent can include a single dose or a series of doses. For example, the method can include administering 1, 2, 3, 4, 5, 6, 7, 8, 9, 10, 15, 20, 25, 30, 35, 40, 45, 50 or more doses of the non-blocking IL-35 binding agent or a pharmaceutical composition comprising the same over the course of treatment. As such, the dose can be administered at a frequency sufficient to produce a therapeutic effect and can be varied. For example, the dose can be administered continuously, hourly, daily, weekly, biweekly, monthly, etc. Further, the therapeutically effective amount of the non-blocking IL-35 binding agent can be increased or decreased over the court of treatment.

Formulations for pharmaceutical compositions are well known in the art. For example, Remington's Pharmaceutical Sciences (18^{th} ed., Mack Publishing Co., Eaton, Pa. 1990), describes compositions and formulations suitable for pharmaceutical delivery of one or more non-blocking IL-35 binding agents, such as one or more anti-IL-35 antibodies and/or small molecules combined with a pharmaceutically acceptable carrier and optionally various pharmaceutically acceptable additives, as well as a dispersion base or vehicle.

The pharmaceutical compositions can be used for oral, rectal, topical, intranasal, transmucosal and parenteral (i.e., subcutaneous, intravenous, intraperitoneal, intramuscular, intraperitoneal, intrasternal or intraarticular) administration, as well as administration through inhaling, although the most suitable route in any given case will depend on the particular subject, and the nature and severity of the immune disorder for which the pharmaceutical composition is being administered. The pharmaceutical compositions may be conveniently presented in unit dosage form and prepared by any method well known in the art of pharmacy.

One type of non-blocking IL-35 binding agent can be anti-IL-35 antibodies. As used herein, “antibody” or “antibodies” means an immunoglobulin molecule immunologically reactive with a particular antigen or epitope of the antigen. The term also includes genetically engineered forms such as chimeric antibodies (e.g., comprising non-human variable regions and human constant regions), humanized antibodies (e.g., comprising non-human variable complementarity determining regions (CDRs) and human variable framework regions (FRs)), as well as fully human antibodies derived from human germline sequences. The term also includes heteroconjugate antibodies (e.g., bispecific antibodies), bivalent or bispecific molecules, diabodies, triabodies and tetrabodies. Bivalent and bispecific molecules are described in, e.g., Kostelny et al. (1992) J. Immunol. 148:1547-1553 (1992); Pack & Pluckthun (1992) Biochemistry 31:1579-1584; Zhu et al. (1997) Protein Sci. 6:781-788; Hu et al. (1996) Cancer Res. 56:3055-3061; Adams et al. (1993) Cancer Res. 53:4026-4034; and McCartney et al. (1995) Protein Eng. 8:301-314; each of which is incorporated herein by reference as if set forth in its entirety.

Antibody also includes antigen-binding forms of antibodies, including fragments with antigen-binding capability (e.g., Fab′, F(ab′)₂, Fab, Fv and rIgG). Treatment of antibodies with proteolytic enzymes, such as papain and pepsin, generates these antibody fragments, especially anti-IL-35 fragments. Antibody also refers to recombinant single chain Fv fragments (scFv). Preferably, antibodies employed to practice the methods described herein bind to its target protein with an affinity (association constant) of equal to or greater than 10⁷ M⁻¹.

The antibody can be a monoclonal or polyclonal antibody and can belong to any antibody class (i.e., IgG, IgM, IgA, etc.). One of ordinary skill in the art is familiar with methods for making monoclonal antibodies (Mab). For example, one of ordinary skill in the art can make monoclonal antibodies by isolating lymphocytes and fusing them with myeloma cells, thereby producing hybridomas. See, e.g., Milstein, In: Handbook of experimental immunology. (Blackwell Scientific Pub., 1986); and Goding, In: Monoclonal antibodies: principles and practice. (Academic Press, 1983); each of which is incorporated herein by reference as if set forth in its entirety. The cloned hybridomas are then screened for production of, e.g., “anti-IL-35” (i.e., antibodies that bind preferentially to IL-35 or fragments thereof). Monoclonal antibodies are thus not limited by the manner in which the antibodies are produced, whether such production is in situ or not.

Alternatively, antibodies can be produced by recombinant DNA technology including, but not limited, to expression in bacteria, yeast, insect cell lines or mammalian cell lines. For example, one or ordinary skill in the art can readily isolated and sequence a nucleic acid sequence encoding a monoclonal antibody using conventional procedures (e.g., by using oligonucleotide probes that are capable of binding specifically to genes encoding the heavy and light chains of an antibody). Hybridoma cells can serve as a preferred source of DNA for the nucleic acid sequence. Once isolated, the nucleic acid sequence can be placed into expression vectors, which are then transfected into host cells such as E. coli cells, simian COS cells, Chinese Hamster Ovary (CHO) cells or myeloma cells that do not otherwise produce antibodies, to obtain the synthesis of monoclonal antibodies in the recombinant host cells. Review articles on recombinant expression in bacteria of DNA encoding an antibody include the following: Skerra (1993) Curr. Opin. Immunol. 5:256-262; and Phickthun (1992) Immunol. Rev. 130:151-188; each of which is incorporated herein by reference as if set forth in its entirety.

Alternatively, antibodies can be produced in a cell line such as a CHO cell line. See, U.S. Pat. Nos. 5,545,403; 5,545,405 and 5,998,144; each of which is incorporated herein by reference as if set forth in its entirety. Briefly, one of ordinary skill in the art can transfect the cell line with vectors capable of expressing a light chain and a heavy chain, respectively. By transfecting the two proteins on separate vectors, chimeric antibodies can be produced. Another advantage of using CHO cells is the correct glycosylation of the antibody.

Likewise, one of ordinary skill in the art is familiar with methods of making polyclonal antibodies. For example, one of ordinary skill in the art can make polyclonal antibodies by immunizing a suitable host animal, e.g., such as a rabbit, with an immunogen and using properly diluted serum or isolating immunoglobulins from the serum. The animal may therefore be inoculated with the immunogen, with blood subsequently being removed from the animal and an IgG fraction purified. Other suitable host animals include a chicken, goat, sheep, guinea pig, rat or mouse. If desired, the immunogen can be administered as a conjugate in which the immunogen is coupled, e.g., via a side chain of one of its amino acid residues, to a suitable carrier. The carrier molecule is typically a physiologically acceptable carrier. The antibody obtained can be purified to a purity of up to about 70%, up to about 80%, up to about 90%, up to about 95%, up to about 99% or up to about 100%.

Methods of making anti-IL-35 antibodies (including, e.g., anti-EBI3 or anti-p35 antibodies) are described in WO 2008/036973, supra. Likewise, commercially available anti-IL-35 antibodies are suitable for use herein, and can be obtained from eBioscience (San Diego, Calif.). For example, anti-p35 antibody clone C18.2 from eBioscience can be used. Also contemplated for use herein are antibodies that bind the same epitope as the commercially available antibodies.

Alternatively, and as noted above, the non-blocking IL-35 binding agent can be a protein designed to bind IL-35 or one of its subunits. As used herein, a “protein designed to bind IL-35” means a protein designed to bind IL-35 or one of its subunits that potentiates IL-35's activity. Such proteins, however, do not block IL-35 from binding to its target(s). For example, one of skill in the art readily can identify a protein designed to bind IL-35 generated by affinity maturation/selection such as from a phage display. See, e.g., Smith (1985) Science 228:1315-1317; incorporated herein by reference as if set forth in its entirety.

Alternatively still, and as noted above, the non-blocking IL-35 binding agent can be a small molecule that binds IL-35 or one of its subunits. As used herein, a “small molecule that binds to IL-35” means a molecule of a size comparable to those molecules generally used in pharmaceuticals that potentiates IL-35's activity, but does not block IL-35 from binding to its target(s). Preferred small organic molecules range in size up to about 5000 Da, more preferably up to about 2000 Da and most preferably up to about 1000 Da. The small molecule can enhance protein-protein interactions between a protein (both membrane bound and soluble) and its receptor, such as between the IL-35 heterodimer and its receptor.

Non-limiting examples of small molecules for use herein include chemical compounds, inorganic molecules, organic molecules, organic molecules containing an inorganic component, molecules including a radioactive atom, synthetic molecules and peptidomimetics (e.g., short, peptide fragments that mimic the most common peptide motifs, such as an α-helix or β-sheet). As the non-blocking IL-35 binding agent, the small molecule may be more permeable to cells, less susceptible to degradation and less apt to elicit an undesired immune response than large molecules.

Regardless of the exact nature of the non-blocking IL-35 binding agent, the composition also can include an effective amount or therapeutically effective amount of IL-35. The exogenous IL-35 and the IL-35 binding agent can be linked via covalent or non-covalent interactions thereby forming a complex. Thus, the various composition and methods described herein can employ the non-blocking IL-35 binding agent or an IL-35/non-blocking IL-35 blocking agent complex.

When the composition is a pharmaceutical composition, it also can include a pharmaceutically acceptable carrier. As used herein, a “pharmaceutically acceptable carrier” means a material that is not biologically, physiologically or otherwise undesirable, i.e., the material can be administered to a subject in a formulation or composition without causing any undesirable biological or physiological effects or interacting in a deleterious manner with any of the components of the composition in which it is contained.

The pharmaceutically acceptable carrier employed can be a solid, liquid or gas. Examples of solid carriers include, but are not limited to, lactose, terra alba, sucrose, talc, gelatin, agar, pectin, acacia, magnesium stearate and stearic acid. Examples of liquid carriers include, but are not limited to, sugar syrup, peanut oil, olive oil, water and saline. Examples of gaseous carriers include, but are not limited to, carbon dioxide and nitrogen.

In addition to the pharmaceutically acceptable carrier, the pharmaceutical compositions can include, as appropriate, one or more additional additives such as diluents, buffers, flavoring agents, binders, surface-active agents, thickeners, lubricants, preservatives (including anti-oxidants) and the like. Moreover, other adjuvants can be included to render the formulation isotonic with the blood of the subject for intravenous administration.

Desired additives include, but are not limited to, pH control agents, such as arginine, sodium hydroxide, glycine, hydrochloric acid, citric acid and the like. In addition, local anesthetics (e.g., benzyl alcohol), isotonizing agents (e.g., sodium chloride, mannitol or sorbitol), adsorption inhibitors (e.g., Tween 80), solubility enhancing agents (e.g., cyclodextrins and derivatives thereof), stabilizers (e.g., serum albumin), reducing agents (e.g., glutathione) and preservatives (e.g., antimicrobials and antioxidants) can be included.

Pharmaceutical compositions for oral dosage can be prepared in any form known in the art. For example, water, glycols, oils, alcohols, flavoring agents, preservatives, coloring agents and the like may be used to form oral liquid preparations such as suspensions, elixirs and solutions, while carriers such as starches, sugars, microcrystalline cellulose, diluents, granulating agents, lubricants, binders, disintegrating agents and the like may be used to form oral solid preparations such as powders, capsules and tablets.

In tablets, the non-blocking IL-35 binding agent can be prepared by compression or molding, optionally with one or more accessory ingredients or adjuvants. Compressed tablets may be prepared by compressing, in a suitable machine, the non-blocking IL-35 binding agent in a free-flowing form such as powder or granules, optionally mixed with a binder, lubricant, inert diluent, surface active or dispersing agent or other such excipient. These excipients can be, e.g., inert diluents such as calcium carbonate, sodium carbonate, lactose, calcium phosphate or sodium phosphate; granulating and disintegrating agents, e.g., corn starch or alginic acid; binding agents, e.g., starch, gelatin or acacia; and lubricating agents, e.g., magnesium stearate, stearic acid or talc. The tablets can be uncoated, or they can be coated by known techniques to delay disintegration and absorption in the gastrointestinal tract and thereby provide a sustained action over a longer time, especially for treating immune system disorders such as inflammatory bowel disease (IBD) or irritable bowel syndrome (IBS). For example, a time delay material such as glyceryl monostearate or glyceryl distearate can be used.

In hard gelatin capsules, the non-blocking IL-35 binding agent can be mixed with an inert, solid diluent, e.g., calcium carbonate, calcium phosphate or kaolin. Conversely, in soft gelatin capsules, the non-blocking IL-35 binding agent can be mixed with water or an oil medium, e.g., peanut oil, liquid paraffin or olive oil. Molded tablets can be made by molding in a suitable machine, a mixture of powdered non-blocking IL-35 binding agent moistened with an inert liquid diluent.

Pharmaceutical compositions for parenteral administration can be prepared as solutions or suspensions of the non-blocking IL-35 binding agents in water. A suitable surfactant can be included such as, e.g., hydroxypropylcellulose. Pharmaceutical compositions can also be prepared in glycerol, liquid polyethylene glycols and mixtures thereof in oils. Alternatively, the pharmaceutical compositions can be prepared in liposomes. See, e.g., Langer (1990) Science 249:1527-1533; and Treat et al. In: Liposomes in the therapy of infectious disease and cancer. (Lopez-Berestein & Fidler, eds.; Liss, N.Y.; 1989. pp. 353-365). Moreover, a preservative can be included to prevent the detrimental growth of microorganisms.

Likewise, pharmaceutical compositions for injection can be prepared as sterile aqueous solutions or dispersions. Alternatively, the compositions can be in the form of sterile powders for sterile injectable solutions or dispersions. The final injectable form must be sterile and must be effectively fluid for easy administration. The pharmaceutical compositions must be stable under the conditions of manufacture and storage and thus, preferably should be preserved against the contaminating action of microorganisms such as bacteria and fungi. As such, the pharmaceutically acceptable carrier can be a solvent or dispersion medium containing, e.g., water, ethanol, polyol (e.g., glycerol, propylene glycol and liquid polyethylene glycol), vegetable oils and suitable mixtures thereof.

Pharmaceutical compositions for topical administration can be prepared, e.g., as an aerosol, cream, ointment, lotion, dusting powder or the like. Alternatively, the pharmaceutical compositions can be in a form suitable for use in transdermal devices. These pharmaceutical compositions may be prepared by methods well known in the art. For example, a cream or ointment can be prepared by admixing water, together with about 5 wt % to about 10 wt % of the binding agent, to produce a cream or ointment having a desired consistency.

Pharmaceutical compositions for rectal administration can be prepared with a solid pharmaceutically acceptable carrier. Preferably, the mixture forms unit dose suppositories. Suitable pharmaceutically acceptable carriers include cocoa butter and other thickening agents commonly used in the art. Suppositories can be conveniently formed by first admixing the composition with a softened or melted pharmaceutically acceptable carrier followed by chilling and shaping in molds.

Pharmaceutical compositions for inhaled administration can be prepared in forms and utilizing carriers known in the art. See, e.g., Zeng et al. In: Particulate interactions in dry powder formulations for inhalation. (Informa HealthCare, 1^{st} ed.; 2000).

Methods

The present invention also includes methods of potentiating IL-35's activity in vitro or in vivo by contacting an effective amount of any of the non-blocking IL-35 binding agents described herein with IL-35, which ultimately affects T_{reg} and/or T_{eff} cells.

As used herein, “regulatory T cell,” “regulatory T cells,” “T_{reg} cell” or “T_{reg} cells” means T lymphocytes that express at least CD4, CD25 and Foxp3 and that secrete IL-35. T_{reg} cells function to suppress or modulate activation of the immune system and thereby maintain immune system homeostasis and tolerance to self-antigens.

As used herein, “effector T cell,” “effector T cells,” “T_{eff} cell” or “T_{eff} cells” means T lymphocytes that express at least CD4 and that secrete interleukin-2 (IL-2), interleukin-4 (IL-4) and/or interferon gamma (IFN-γ). T_{eff} cells do not secrete IL-35 and lack endogenous cytotoxic or phagocytic activity. T_{eff} cells function to regulate or assist other T cells in an immune response.

In one embodiment, a method of enhancing IL-35 activity in vitro is provided, which comprises contacting an effective amount of a non-blocking IL-35 binding agent with IL-35. The non-blocking IL-35 binding agent potentiating IL-35, thereby enhancing IL-35 activity. In addition, a method of enhancing IL-35 activity in vivo is provided, which comprises administering to a subject a therapeutically effective amount of a non-blocking IL-35 binding agent that increases the half-life of IL-35. The non-blocking IL-35 binding agent potentiating IL-35, thereby enhancing IL-35 activity.

In another embodiment, a method of enhancing immune suppression is provided, which comprises administering to a subject having, or susceptible to having, an immune system disorder a therapeutically effective amount of a non-blocking IL-35 binding agent. The non-blocking IL-35 binding agent potentiating IL-35, thereby enhancing immune suppression.

In another embodiment, a method of attenuating T_{eff} cells is provided, which comprises administering to a subject having, or susceptible to having, aberrant T_{eff} cell function a therapeutically effective amount of a non-blocking IL-35 binding agent. The non-blocking IL-35 binding agent potentiating IL-35, thereby attenuating T_{eff} cell function. As used herein, “aberrant T_{eff} cell function” means that T_{eff} cells having an increased involvement in activating and directing other immune cells. For example, aberrant T_{eff} cell function includes, but is not limited to, increased B cell antibody class switching, increased activation and growth of cytotoxic T (T_{cyto}) cells, and increased stimulation of bactericidal activity of phagocytes such as macrophages and other immune cells. Overall, “aberrant T_{eff} cell function” means an up-regulation of those parts of the immune system influenced by T_{eff} cells.

In yet another embodiment, a method of expanding T_{reg} cells is provided, which comprises administering to a subject having, or susceptible to having low T_{reg} cell numbers a therapeutically effective amount of a non-blocking IL-35 binding agent. The non-blocking IL-35 binding agent potentiating IL-35, thereby expanding T_{reg} cells. As used herein, “expanding T_{reg} cells” means that naïve T_{eff} cells are converted to T_{reg} cells or that regulatory activity is conferred upon naïve T_{eff} cells.

In some embodiments, the subject can be a mammal, such as a primate, including a human, or a domestic or agricultural animal. In those embodiments in which the subject has an immune system disorder, the disorder can be an autoimmune condition or inflammatory condition.

In other embodiments, exogenous IL-35 can be administered with the non-blocking IL-35 binding agent or IL-35/non-blocking IL-35 binding agent complex.

The therapeutically effective amount of the non-blocking IL-35 binding agent can be administered at about the same therapeutically effective amount (i.e., dose) throughout a treatment period, in an escalating dose regimen or loading-dose regime (e.g., in which the loading dose is about two to five times the maintenance dose). Alternatively, the therapeutically effective amount can be varied during the course of a treatment based on the condition of the subject being treated, the apparent response to the therapy, and/or other factors as judged by one of ordinary skill in the art. Long-term treatment with the therapeutically effective amount is also contemplated.

The methods described herein are directed at potentiating IL-35's activity with the non-blocking IL-35 binding agent, as opposed to suppressing or attenuating its activity. The non-blocking IL-35 binding agent therefore can be provided in vitro or in vivo to increase IL-35's t_{1/2}, to potentiate IL-35's activities such as expanding T_{reg} cells, conferring regulatory activity on naïve T cells or attenuating T_{eff} cell function.

The methods therefore find particular use in treating immune system disorders in which enhanced immune suppression is desired, such as in autoimmune conditions and inflammatory conditions. Examples of autoimmune conditions include, but are not limited to, acute disseminated encephalomyelitis (ADEM), Addison's disease, Alopecia areata, ankylosing spondylitis (AS), anti-phospholipid antibody syndrome (APS), autoimmune hemolytic anemia, autoimmune hepatitis, autoimmune inner ear disease, Bullous pemphigoid (BP), celiac disease, chronic obstructive pulmonary disease (COPD), Crohn's disease, dermatomyositis, diabetes mellitus type I, endometriosis, fibromyalgia, Goodpasture's syndrome, Graves' disease, Guillain-Barré syndrome (GBS), Hashimoto's thyroiditis, idiopathic thrombocytopenic purpura (ITP), interstitial cystitis, systemic lupus erythematosus (SLE), multiple sclerosis (MS), myasthenia gravis, pernicious anemia, polymyositis, primary biliary cirrhosis, rheumatoid arthritis, schizophrenia, scleroderma, Sjögren's syndrome, ulcerative colitis, vasculitis, vitiligo and Wegener's granulomatosis. Likewise, examples of inflammatory conditions include, but are not limited to, allergy, asthma, transplant rejection, cancer, inflammatory bowel disease (IBD), inflammatory bowel syndrome (IBS), Chagas disease, psoriasis, keloid, atopic dermatitis, lichen simplex chronicus, prurigo nodularis, Reiter syndrome, pityriasis rubra pilaris, pityriasis rosea, stasis dermatitis, rosacea, acne, lichen planus, scleroderma, seborrheic dermatitis, granuloma annulare, rheumatoid arthritis, dermatomyositis, alopecia areata, lichen planopilaris, vitiligo and discoid lupus erythematosis. To be clear, some of the immune disorders listed above can be classified as both an autoimmune condition and an inflammatory condition.

As used herein, “enhancing immune suppression” means decreasing an ability of a cell or subject, particularly a mammal, to initiate or sustain an immune response or downregulating an immunostimulatory capacity of the cell or subject, and the like.

Administration can begin when the subject is diagnosed with the immune system disorder or is suspected of having the immune system disorder. Acceptable therapeutically effective amounts of the non-binding IL-35 binding agent are discussed above and will vary depending upon the subject, the immune system disorder being treated and the route of administration. The method may comprise a single administration of the therapeutically effective amount or multiple administrations of the therapeutically effective amount of the non-blocking IL-35 binding agent.

The effects of the non-blocking IL-35 binding agent on IL-35 activity can be evaluated by any method known in the art. Of particular interest are those methods of assaying T cell (i.e., T_{reg} and T_{eff} cells) concentration and function. For example, a white blood cell count (WBC) can be used to determine the responsiveness of a subject's immune system. The WBC measures the number of white blood cells in the subject. Using methods well known in the art, the white blood cells in the subject's blood sample are separated from other blood cells and counted. Normal values of white blood cells are about 4,500 to about 10,000 white blood cells/μl. Lower numbers of white blood cells can be indicative of a state of immunosuppression in the subject. Alternatively, immunosuppression in the subject can be determined by way of a T lymphocyte count. T lymphocytes are differentiated from other white blood cells using standard methods in the art, such as, e.g., immunofluorescence or fluorescence activated cell sorting (FACS). Reduced numbers of T cells, or a specific population of T cells (e.g., T_{eff} or CD8⁺ T cells) can be used as a measurement of immunosuppression. A reduction in the number of T cells, or in a specific population of T cells, compared to the number of T cells (or the number of cells in the specific population) prior to a specific event can be used to indicate that immunosuppression has been induced. Conversely, one could measure for proliferating T_{reg} cells.

Methods for isolating and quantifying of T_{reg} cells, such as CD4⁺Foxp3⁺ T_{reg} cells, and other populations of T cells (e.g., T_{eff} or CD8⁺ cells), are well known in the art. Typically, labeled antibodies specifically directed to one or more cell surface markers are used to identify and quantify the T cell population. The antibodies can be conjugated to other compounds including, but not limited to, enzymes, magnetic beads, colloidal magnetic beads, haptens, fluorochromes, metal compounds, radioactive compounds or drugs. The enzymes that can be conjugated to the antibodies include, but are not limited to, alkaline phosphatase, peroxidase, urease and β-galactosidase. The fluorochromes that can be conjugated to the antibodies include, but are not limited to, fluorescein isothiocyanate (FITC), tetramethylrhodamine isothiocyanate, phycoerythrin (PE), allophycocyanins and Texas Red. For additional fluorochromes that can be conjugated to antibodies, see, Haugland, In: Handbook of fluorescent probes and research products. (Molecular Probes, 9^{th} ed.; 2002). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone and nitrophenol. The radioactive compounds that can be conjugated or incorporated into the antibodies are known to the art, and include, but are not limited to, ⁹⁹Tc, ¹²⁵I, and amino acids comprising any radionuclides, including, but not limited to, ¹⁴C, ³H and ³⁵S.

FACS also can be used to sort cells that are CD4⁺, CD25⁺, both CD4⁺ and CD25+, or CD8⁺ by contacting the cells with an appropriately labeled antibody. However, other techniques of differing efficacy may be employed to purify and isolate desired populations of cells. The separation techniques employed should maximize the retention of viability of the fraction of the cells to be collected. The particular technique employed will, of course, depend upon the efficiency of separation, cytotoxicity of the method, the ease and speed of separation, and what equipment and/or technical skill is required.

Additional separation procedures may include magnetic separation, using antibody-coated magnetic beads, affinity chromatography, cytotoxic agents, either joined to a monoclonal antibody or used in conjunction with complement, and “panning,” which utilizes a monoclonal antibody attached to a solid matrix, or another convenient technique. Antibodies attached to magnetic beads and other solid matrices, such as agarose beads, polystyrene beads, hollow fiber membranes and plastic Petri dishes, allow for direct separation. Cells that are bound by the antibody can be removed from the cell suspension by simply physically separating the solid support from the cell suspension. The exact conditions and duration of incubation of the cells with the solid phase-linked antibodies will depend upon several factors specific to the system employed. The selection of appropriate conditions, however, is well known in the art.

Unbound cells then can be eluted or washed away with physiologic buffer after sufficient time has been allowed for the cells expressing a marker of interest (e.g., CD4 and/or CD25) to bind to the solid-phase linked antibodies. The bound cells are then separated from the solid phase by any appropriate method, depending mainly upon the nature of the solid phase and the antibody employed, and quantified using methods well known in the art. In one example, bound cells separated from the solid phase are quantified by FACS. Antibodies may be conjugated to biotin, which then can be removed with avidin or streptavidin bound to a support, or fluorochromes, which can be used with FACS to enable cell separation and quantification, as known in the art.

In yet another embodiment, a method to identify a non-blocking IL-35 binding agent is provided, which comprises contacting IL-35 with a candidate agent suspected of binding IL-35 to form an IL-35/candidate agent complex. One then determines whether the IL-35/candidate agent complex formed and whether it potentiates activity of IL-35. The IL-35/candidate agent complex can be directly or indirectly detected. Likewise, IL-35 activity can be determined by assaying for a potentiated IL-35 signal or by assaying for increased IL-35 t 1/2.

In a related embodiment, a method to assay an ability of a known IL-35 binding agent to potentiate IL-35 is provided, which comprises determining whether an IL-35/known binding agent complex potentiates activity of IL-35. IL-35 activity can be determined by assaying for a potentiated IL-35 signal or by assaying for increased IL-35 t½.

The likelihood of an assay identifying a small molecule that acts as a non-blocking IL-35 binding agents increases when the number and types of test agents used in a screening system is increased. Recently, attention has focused on the use of combinatorial chemical libraries to assist in the generation of new small molecule inhibitor leads. A combinatorial chemical library is a collection of diverse chemical compounds generated by either chemical synthesis or biological synthesis by combining a number of chemical “building blocks.” For example, a linear combinatorial chemical library, such as a polypeptide library, is formed by combining a set of chemical building blocks (e.g., amino acids) in every possible way for a given compound length (i.e., the number of amino acids in a polypeptide compound). Millions of chemical compounds can be synthesized through such combinatorial mixing of chemical building blocks (see, e.g., Gallop et al. (1994) J. Med. Chem. 37:1233-1250).

The putative non-blocking IL-35 binding agents employed in the screening assay can include any candidate agent compound including, but not limited to, peptides, peptidomimetics, small molecules, antibodies or even drugs. Such putative non-blocking IL-35 binding agents can be obtained using any combinatorial library method known in the art including, but not limited to, biological libraries, spatially addressable parallel solid phase or solution phase libraries, synthetic libraries and the like.

Preparation and screening of combinatorial chemical libraries is well known to one of ordinary skill in the art. Such combinatorial chemical libraries include, e.g., peptide libraries (see, e.g., U.S. Pat. No. 5,010,175). Peptide synthesis is not the only approach envisioned and intended for use herein. Other chemistries for generating chemical diversity libraries can also be used. Examples of such chemistries include, but are not limited to, peptoids (see, e.g., WO 91/19735), encoded peptides (see, e.g., WO 93/20242), random bio-oligomers (see, e.g., WO 92/00091), benzodiazepines (see, e.g., U.S. Pat. No. 5,288,514), diversomers such as hydantoins, benzodiazepines and dipeptides (see, e.g., DeWitt et al. (1993) Proc. Nat. Acad. Sci. USA 90:6909-6913), vinylogous polypeptides (see, e.g., Hagihara et al., (1992) J. Amer. Chem. Soc. 114:6568-6570), nonpeptidal peptidomimetics with a β-D-glucose scaffolding (see, e.g., Hirschmann et al. (1992) J. Amer. Chem. Soc. 114:9217-9218), analogous organic syntheses of small compound libraries (see, e.g., Chen et al. (1994) J. Amer. Chem. Soc. 116:2661-2662), oligocarbamates (see, e.g., Cho et al. (1993) Science 261:1303-1305), and peptidyl phosphonates (see, e.g., Campbell et al. (1994) J. Org. Chem. 59:658-660). In addition, a number of combinatorial libraries are commercially available, as is well known to one of ordinary skill in the art. High throughput techniques can be used to screen any of the various libraries described herein. As is also well known to one of ordinary skill in the art, a number of high throughput screening systems are commercially available (e.g., Zymark Corp., Hopkinton, Mass.; Air Technical Industries, Mentor, Ohio; Beckman Instruments, Inc., Fullerton, Calif.; and Precision Systems, Inc., Natick, Mass.). These systems typically automate entire procedures including all sample and reagent pipetting, liquid dispensing, timed incubations and final readings of a microplate in detector(s) appropriate for the assay. These configurable systems provide high throughput and rapid start up as well as a high degree of flexibility and customization.

The methods of screening described herein are directed at discovering non-blocking IL-35 binding agents that potentiate IL-35's activity. One can screen for non-blocking IL-35 binding agents by contacting a putative non-blocking IL-35 binding agent with IL-35.

One can determine whether a non-blocking IL-35 binding agent/IL-35 complex formed by any of the various direct or indirect methods known in the art. For example, one can couple the putative non-blocking IL-35 binding agent to a radioisotope, enzymatic label or fluorescent label. Examples of radioisotopes for use with the methods herein include, but are not limited to, ¹²⁵I, ³⁵S, ¹⁴C or ³H, either directly or indirectly. The radioisotope can be detected by radioemmission or scintillation counting. Likewise, examples of enzymatic labels for use with the methods herein include, but are not limited to, horseradish peroxidase, alkaline phosphatase or luciferase. The enzymatic label can be detected by conversion of appropriate substrate to product.

One can determine whether the non-blocking IL-35 binding agent potentiates IL-35 activity by any of the various IL-35 activity assays known in the art. For example, one can use the methods described by Collison et al., supra; Niedbala et al., supra; and WO 2008/036973, supra, each of which is incorporated herein by reference as if set forth in its entirety.

The invention will be more fully understood upon consideration of the following non-limiting Examples.

### EXAMPLES

### Example 1

### Non-Blocking Anti-IL-35 Antibodies Potentiate IL-35 Inhibition of Effector T Cells

This examples shows that anti-EBI3 and anti-p35 antibodies can effectively modulate the activity of IL-35.

Methods.

Proliferation assay: FACS-purified T_{eff} cells (2.5×10⁴/well) were pre-incubated with indicated antibodies at 10 μg/ml for 10 minutes at 37° C. Following antibody treatment, T_{eff} cells were activated with anti-CD3⁺ anti-CD28 coated sulfate latex beads at 5×10³/well in the presence of dialyzed, filtered HEK293T supernatant containing rIL-35. Proliferation was determined by [3H]-thymidine incorporation.

In a second proliferation assay, T_{eff} cells (2.5×10⁴/well) were activated in the presence of rIL-35 containing supernatant and antibodies at 2.5, 5 and 10 μg/ml. IgG1 and IgG2 isotype controls were used to determine specificity. Proliferation was determined by [3H]-thymidine incorporation.

Results.

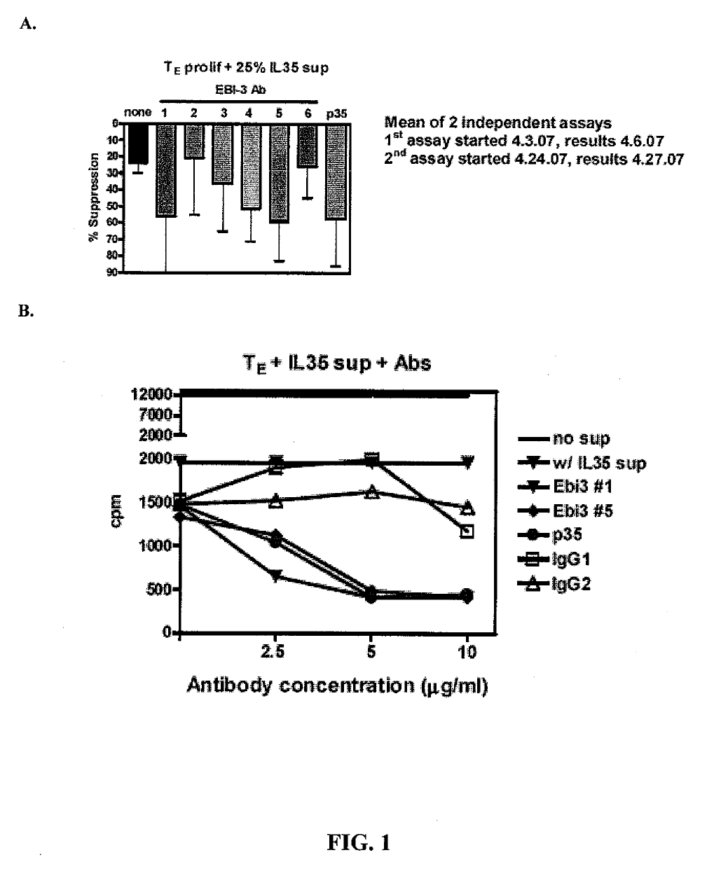

FIG. 1A shows that anti-EBI3 antibodies 1, 4 and 5, as well as anti-p35 antibody (clone C 18.2 from eBioscience), significantly potentiated the suppressive capacity of IL-35 upon the proliferation of T_{eff} cells. Similarly, FIG. 1B shows that further analysis of anti-EBI3 antibodies 1 and 5, as well as the anti-p35 antibody, potentiated the suppressive capacity of IL-35 in a dose-dependent manner. Antibody isotype controls had no effect of IL-35 suppression.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

1. A method of potentiating IL-35 activity, the method comprising the step of:
contacting IL-35 with an effective amount of a non-blocking interleukin 35 (IL-35) binding agent, wherein the non-blocking IL-35 binding agent potentiates IL-35 activity, and wherein the non-blocking IL-35 binding agent is an antibody.

2. The method of claim 1, wherein the antibody is a monoclonal antibody.

3. The method of claim 1, wherein the antibody binds to an Epstein Barr virus induced gene 3 (EBI3) chain of IL-35.

4. The method of claim 1, wherein the antibody binds to an interleukin 12a (IL-12a)/p35 chain of IL-35.

5. The method of claim 1, wherein the non-blocking IL-35 binding agent is provided in vivo to a subject having or susceptible to having an immune system disorder.

6. The method of claim 5, further comprising the step of providing a therapeutically effective amount of IL-35 to the subject.

7. The method of claim 5, wherein the immune system disorder is selected from the group consisting of an autoimmune condition and an inflammatory condition.

8. The method of claim 5, wherein the subject is a mammal.

9. The method of claim 8, wherein the mammal is a primate.

10. The method of claim 9, wherein the primate is a human.

11. The method of claim 1, wherein potentiated IL-35 activity results in enhanced regulatory T cell function.

12. The method of claim 1, wherein potentiated IL-35 activity results in regulatory T cell expansion.

13. The method of claim 1, wherein potentiated IL-35 activity results in attenuated effector T cell function.

14. A method of increasing interleukin 35 (IL-35) half-life, the method comprising the step of:
contacting IL-35 with an effective amount of a non-blocking IL-35 binding agent, wherein the non-blocking IL-35 binding agent increases IL-35 half-life, and wherein the non-blocking IL-35 binding agent is an antibody.

15-26. (canceled)

27. A method of attenuating effector T cell function, the method comprising the step of:
contacting IL-35 with an effective amount of a non-binding interleukin 35 (IL-35) binding agent in the presence of a T cell population, wherein the non-blocking IL-35 binding agent potentiates IL-35 activity, and wherein the non-blocking IL-35 binding agent is an antibody.

28-35. (canceled)

36. A method of expanding regulatory T cells, the method comprising the step of:
contacting IL-35 with an effective amount of a non-binding interleukin 35 (IL-35) binding agent in the presence of a T cell population, wherein the non-blocking IL-35 binding agent potentiates IL-35 activity, and wherein the non-blocking IL-35 binding agent is an antibody.

37-44. (canceled)

45. A method to identify a non-blocking interleukin 35 (IL-35) binding agent that enhances IL-35 activity, the method comprising the steps of:
contacting IL-35 with a candidate non-blocking IL-35 binding agent;
determining whether the candidate agent and IL-35 formed a candidate agent/IL-35 complex; and
determining whether the candidate agent/IL-35 complex enhances IL-35 activity.

46. The method of claim 45, wherein determining whether the candidate agent//IL-35 complex formed comprises (1) directly detecting the candidate agent/IL-35 complex, or (2) indirectly detecting the candidate agent/IL-35 complex using a competitive binding assay.

47. The method of claim 45, wherein determining whether the candidate agent/IL-35 complex enhances IL-35 activity comprises assaying for a potentiated IL-35 activity or assaying for increased half-life of IL-35.
